(19) United States
(12) Patent Application Publication
(10) Pub. No.: US 2005/0267197 A1
(43) Pub. Date: Dec. 1, 2005

(54) Compositions containing policosanol and HMG-Co-A reductase inhibitor and their pharmaceutical uses

(76) Inventor: Roger Berlin, Mendham, NJ (US)

Correspondence Address: WYETH;PATENT LAW GROUP, 5 GIRALDA FARMS, MADISON, NJ, 07940, US

(21) Appl. No.: 10/853,578

(22) Filed: May 25, 2004

(51) Int. Cl. A61K 31/401; A61K 31/366; A61K 31/045; A61K 31/225

(52) U.S. Cl. 514/423; 514/460; 514/548; 514/724

(57) ABSTRACT

A composition is provided which contains policosanol and HMG-Co-A reductase inhibitor and which may be used for treating and or reducing hypercholesterolemic diseases, total cholesterol, LDL-cholesterol, coronary heart disease (heart attacks and strokes), inflammation, deep-vein thrombosis immunoregulatory diseases, cardiovascular diseases, and/or neurodegenerative disorders in humans and animals. The method comprises administering policosanol and HMG-Co-A reductase inhibitor which together effectively lower serum cholesterol levels. Typically, the administered composition includes about 0.1-10:1 parts by weight of policosanol to HMG-Co-A reductase inhibitor.

### BACKGROUND OF THE INVENTION

1. Field of the Invention

The present invention relates to therapeutic compositions and methods for reducing serum cholesterol levels in humans and animals. More particularly the invention pertains to a therapeutic composition and method for reducing serum cholesterol levels by administering a composition comprising a biologically active mixture of high purity, high molecular weight straight chain primary aliphatic alcohols (referred to collectively herein as policosanol) and a 3-hydroxy-3-methylglutaryl co-enzyme A (HMG-CoA) reductase inhibitor.

2. Description of the State of Art

According to the American Heart Association (AHA), about 62 million Americans have some form of cardiovascular disease, which can include high blood pressure, coronary heart disease (heart attack and chest pain), stroke, birth defects of the heart and blood vessels, and congestive heart failure, and close to a million die from such conditions every year. The annual report of the AHA further states that cardiovascular disease kills more Americans than the next 7 causes of death combined, including cancer. Surprisingly, slightly more females, overall, than males have cardiovascular disease. Heart disease accounted for 40% of all deaths in the U.S. in 1999.

According to the National Heart, Lung, and Blood Institute (NHLBI) the higher your blood cholesterol, the greater your risk for developing heart disease and suffering a heart attack. However, because high blood cholesterol does not cause any symptoms, many people (more than 50 percent by recent estimates) are either inadequately treated or unaware that their cholesterol level is too high. Considering that 41 million estimated American adults have high cholesterol (according to the AHA), the failure to appreciate high cholesterol's importance places many people at unnecessary risk for developing future heart disease.

Cholesterol is a soft waxy, fat-like substance that is necessary for good health. It is a normal component of most body tissues, especially those of the brain, nervous system, muscle, skin, liver, intestines, and heart. Without cholesterol, our bodies could not function properly. It is needed to form the sex and adrenal hormones, vitamin D and bile (a digestive secretion required for fat digestion).

Cholesterol in the body comes from two major sources. The first is from the liver, which is the body's major cholesterol-producing organ. The second source is from eating animal products such as meat (beef, chicken, fish), egg yolks, cheese and other whole milk products. Because the liver is usually able to make enough cholesterol to satisfy all of our bodily needs, too much dietary cholesterol can lead to high bodily levels of cholesterol. These high levels are undesirable because it is difficult for our bodies to appropriately dispose of excess cholesterol.

Cholesterol, triglycerides, and other lipid molecules are transported through the bloodstream by protein spheres called lipoproteins. Most of the information about the effects of cholesterol and triglyceride actually concerns lipoproteins. Lipoproteins are categorized into five types according to size and density. They can be further defined by whether they carry cholesterol (the two smaller lipoproteins) or triglycerides (the three largest lipoproteins).

Cholesterol-carrying lipoproteins (low-density and high-density lipoproteins) are the lipoproteins commonly referred to as cholesterol. Cholesterol also behaves differently depending on which type of lipoprotein carries it. Low Density Lipoprotein (LDL) transports about 75% of the blood's cholesterol to the body's cells. It is normally harmless. However, if it is exposed to a process called oxidation, it can penetrate and interact dangerously with the walls of the artery, producing a harmful inflammatory response. When LDL collects on arterial walls oxidants are produced and released from the wall membranes. These oxidants tend to bind to and modify the LDL, thereby signaling the immune system that a harmful molecule has appeared. In response to oxidized LDL, the body releases various immune factors aimed at protecting the damaged walls. Unfortunately, in excessive quantities they cause inflammation and promote further injury to the areas they target. White blood cells and other factors gather and form the fatty substance called plaque. Over time the growth of plaque on the artery walls narrow the artery and obstructs the flow of blood. This is referred to as atherosclerosis or “hardening of the arteries”. If the blood flow to the heart is blocked, a heart attack can occur. If the blood flow to the brain is blocked, a stroke can occur. Since LDLs promote atherosclerosis, they are known as “bad cholesterol.” The NHLBI classification of the optimal level of LDL cholesterol is less than 100 milligrams (mg) per deciliter (dL). Borderline high is 130-159 mg/dL, and very high is 190 mg/dL and above. High LDL cholesterol always requires attention. Since the majority of cholesterol is in the form of LDLs, a high blood cholesterol level means high LDL levels and the higher the LDL level, the higher the risk of heart problems.

Lipoprotein(a) (Lp(a)) is a type of LDL cholesterol modified by the addition of an apolipoprotein in the liver. There is a significant association between high levels of Lp(a) and an increased risk of cardiovascular disease. The median level of Lp(a) in the general population is 4 mg/dL. About 20% of the population appears to have increased levels of Lp(a), a purely genetic characteristic, and those in the 90th percentile have an average of 18 mg/dL. Lowering a high level of Lp(a) is difficult. The best means of reduction is to decrease LDL cholesterol as much as possible, since lowering LDL cholesterol substantially decreases the risk associated with elevated Lp(a).

High Density Lipoprotein (HDL) or “good cholesterol” actually removes cholesterol from the walls of arteries and brings it back to the liver to be safely excreted. It also helps prevent oxidation of LDL. In fact, it appears to have antioxidant properties on its own. People who exercise, don't smoke, and stay at their ideal weight tend to have higher levels of HDLs. HDL cholesterol protects against heart disease. This means that higher numbers of HDL cholesterol are better. A level less than 40 mg/dL is considered low and a major risk factor for the development of coronary artery disease, whereas HDL levels of 60 mg/dL or more help to lower the risk for heart disease.

The remaining three types of lipoproteins, that is, intermediate density lipoproteins (IDL), very low-density lipoproteins (VLDL), and chylomicrons are triglyceride-carrying lipoproteins. Triglycerides are another type of substance closely related to cholesterol. While less is known about triglycerides, in general, there is some evidence to suggest that they are a particularly important cause of coronary artery disease among women and people with other risk factors such as diabetes and obesity. Triglycerides also can raise heart disease risk. Levels that are borderline high (150-199 mg/dL) or high (200 mg/dL or more) may require treatment for some people.

According to the new guidelines released in May 2001 by the NHLBI's National Cholesterol Education Program (NCEP), everyone age 20 and older should have their cholesterol and triglyceride levels measured at least once every five years. This blood test is done after a 9- to 12-hour fast and provides information about one's total cholesterol (TC), LDL and HDL cholesterol, and triglycerides. If the total blood cholesterol is 200 milligrams (mg) per deciliter (dL) or more, or if the HDL level is less than 40 mg/dL, a physician should be consulted on ways to lower one's total blood cholesterol.

More recently, experts have begun to examine the individual components of the lipid profile, in addition to the total cholesterol level. While an elevated total cholesterol level is a risk factor, the levels of the various forms of cholesterol which make up the total cholesterol may be a better indication of risk factors. For example, studies indicate that the ratio of LDL cholesterol to HDL cholesterol is more important than individual levels of LDL cholesterol and HDL cholesterol in that the ratio is a more accurate measure of risk of cardiovascular disease. The higher the LDL/HDL ratio, the higher the risk of cardiovascular disease. Ideally, the LDL/HDL ratio should not exceed 4.4. An LDL/HDL ratio in the range of 4.4 to 7.1 is considered to indicate an average risk of cardiovascular disease. A moderate risk ratio is 7.1 to 11, and any ratio above 11 is considered to indicate a high risk of cardiovascular disease.

Evidence has been accumulating in recent years that driving cholesterol even lower than the current guidelines recommend may produce additional benefits. However, researchers are hesitant to prescribe higher dosages of the costly drugs until clear evidence has shown it would keep people healthier and reduce their risk of dying.

Lowering blood cholesterol levels is important for everyone, including younger, middle-aged, and older adults, and people with or without heart disease and/or stroke. Lowering blood cholesterol levels that are too high lessens the risk for developing heart disease and reduces the chance of a heart attack or dying of heart disease. This is especially true for people who have already suffered a heart attack. Blood cholesterol levels are affected by many factors, which includes diet, increasing exercise, or medication. This is very important because with every 1 percent reduction in total blood cholesterol, there is about a 2 percent reduction in the risk of heart attack.

When a patient without heart disease is first diagnosed with elevated blood cholesterol, physicians often prescribe a program of diet, exercise, and weight loss to bring levels down. The National Cholesterol Education Program guidelines suggest at least a six-month program of reduced dietary saturated fat and cholesterol, together with physical activity and weight control, as the primary treatment before resorting to drug therapy. Typically, physicians prescribe the Step I/Step II diet devised by the National Institutes of Health, National Heart, Blood and Lung Institute, aimed at lowering LDL cholesterol. The goals of the Step I Diet are to limit cholesterol intake to less than 300 mg per day and fat intake to 30 percent or less of the day's total calories, with only 8 percent to 10 percent of calories from saturated fat. The more aggressive Step II Diet limits cholesterol intake to less than 200 mg per day and fat intake to 30 percent or less of the day's total calories, with less than 7 percent of total calories from saturated fat. Many patients respond well to this diet and end up sufficiently reducing blood cholesterol levels.

People who are on a cholesterol-lowering diet, however, are successful in actually lowering their risk for heart disease only if they also follow a regular aerobic exercise program. Some studies suggest that for the greatest heart protection, it is not the duration of a single exercise session that counts but the total daily amount of energy expended. Therefore, the best way to exercise may be in multiple short bouts of intense exercise. Burning at least 250 calories a day (the equivalent of about 45 minutes of brisk walking or 25 minutes of jogging) seems to confer the greatest protection against coronary artery disease, most likely because it raises HDL levels. Note, however, moderate exercise has little effect on HDL, and it may take up to a year of sustained exercise to make any significant difference on HDL levels.

Aerobic exercise appears to raise HDL levels, open up the blood vessels and, in combination with a healthy diet, may improve blood-clotting factors. Resistance (weight) training offers a complementary benefit to aerobics by reducing LDL levels.

In many cases, a real change in diet along with more physical activity may not be enough to lower elevated LDL cholesterol and raise HDL cholesterol to recommended levels. Drug treatment should be considered for patients who, in spite of dietary changes, regular physical activity and weight loss, need further treatment for an elevated LDL cholesterol level. Perhaps a genetic predisposition to high blood cholesterol exits. In these cases, physicians often prescribe drugs. The National Cholesterol Education Program estimates that as many as 9 million Americans take some form of cholesterol-lowering drug therapy. Currently, there are four main classes of prescription medications available that lower cholesterol. These are the Niacins, Statins, Fibrates, and Resins, each of which is discussed in more detail below.

Medications

Niacins

Niacin (vitamin B3), is the oldest lipid-lowering agent that has been proved to decrease cardiovascular morbidity and total mortality. Niacin is an attractive drug because it is relatively inexpensive and significantly lowers cholesterol levels by reducing the liver's production of LDL cholesterol, Lp(a), and another kind of cholesterol called VLDL cholesterol. Of the prescription medications, only niacin has been shown to reduce Lp(a), making it unique among the lipid-lowering agents. Niacin can reduce total cholesterol by up to 15%. Niacin also increases the blood levels of HDL cholesterol by up to 35%, which is superior to other lipid-lowering agents, which only increase HDL levels up to 15% at best. By selectively decreasing triglyceride synthesis, niacin also reduces triglyceride levels by up to 30%.

Several different preparations of niacin are available: crystalline or immediate release preparations, and controlled/extended/sustained/prolonged release formulations. Controlled/extended/sustained/prolonged release formulations (collectively referred to herein as “controlled release”) are designed to slowly release the active ingredient from the tablet or capsule, which allows a reduction in dosing frequency as compared to the typical dosing frequency associated with conventional or immediate dosage forms. The slow drug release reduces and prolongs blood levels of the drug and, thus, minimizes or lessens the cutaneous flushing side effects that are associated with conventional or immediate release niacin products. While the controlled release preparations are more effective in lowering LDL than the immediate release formulations, the controlled release products do not have the same advantageous lipid-altering effects as immediate release niacin, and in fact have a worse side effect profile. For example, when changes in HDL are examined, immediate release niacin is much more effective in increasing HDL than the controlled release formulations. The controlled release formulation never increases HDL by more than about 10%. Immediate release niacin is also more effective in lowering triglycerides. Additionally, controlled release niacin formulations are known to cause greater incidences of liver toxicity. The characteristics of the newest controlled niacin preparation, Niaspan®, however, is better tolerated and appears to be less harmful on the liver than other controlled release formulations.

Niacin therapy is plagued by low compliance rates due to its negative side effects. Studies indicate that more than 40% of the patients treated with standard niacin discontinue it because of adverse effects. The most common side effect of niacin is flushing (redness) of the face and neck, affecting more than three quarters of patients. Flushing can be minimized by starting the patient on a low dosage of niacin and making small increases periodically. It is also helpful for the patient to take 162 mg to 325 mg of aspirin 30 minutes before the niacin. Taking the niacin at bedtime and avoiding concomitant ingestion of alcohol or hot beverages will further diminish flushing. Despite these tips, many patients are noncompliant with this regimen and fail to continue use of the drug beyond the initial stage of flushing. Other adverse effects include abdominal pain, peptic ulcers, nausea, gas, heartburn, diarrhea, skin rashes, itching and low blood pressure.

Niacin can also raise homocysteine levels, which can increase the risk of heart disease. Abnormal homocysteine levels appear to contribute to atherosclerosis in at least three ways: (1) a direct toxic effect that damages the cells lining the inside of the arteries, (2) interference with clotting factors, and (3) oxidation of low-density lipoproteins (LDL).

Niacin may also promote glucose intolerance and should be used with caution in patients with diabetes mellitus. Niacin can also increase uric acid levels and precipitate a gout attack. Controlled release formulations of niacin tablets are less liable than regular niacin to have these effects on glucose and uric acid.

There have also been reports of liver necrosis with niacin therapy. Controlled release formulations of niacin tablets pose a greater risk than regular niacin to cause liver damage. Liver function must be monitored in any patient who takes niacin. The onset of nausea and malaise in a patient who is taking niacin almost always indicates abnormal liver function.

Statins

The most prominent cholesterol lowering drugs are a class of synthetically derived HMG-CoA reductase inhibitors in the statin family, used to aggressively treat elevated LDL levels. An array of powerful treatments that includes Mevacor® (lovastatin), Lescol® (fluvastatin), Crestor® (rosuvastatin), Pravachol® (pravastatin), Zocor® (simvastatin), Baycol® (cervastatin), and Lipitor® (atorvastatin). These are collectively refered to as “statin” drugs and are among the most potent lipid-lowering agents available. By inhibiting the production of HMG-CoA reductase, cholesterol production in the liver is reduced. To compensate for the resulting reduction of cholesterol production, the liver increases absorption of LDL cholesterol, further contributing to an overall reduction of LDL cholesterol levels. Studies indicate that for every 10 percent reduction of cholesterol, the risk of death from heart disease decreased 15 percent.

While statin drugs are effective in lowering LDL cholesterol, they do have serious side effects. In August 2001, Bayer AG withdrew the cholesterol-lowering statin drug Baycol® from the market because it was linked to 31 deaths. Moreover, deaths occurred at the manufacturer's recommended initial dose (0.4 mg/day) as well as at the highest dose (0.8 mg/day). More recently, in March 2004, a consumer advocacy group sought to have Crestor® banned, citing 16 cases of serious side effects. The majority of deaths occurred in elderly patients and more often in women.

Like Baycol®, other statin drugs on the market, such as Lipitor® (currently the best seller) are linked to the same rare muscle weakness, known as myositis, which occurs in about 1 in 1,000 statin users. Myositis occasionally progresses to rhabdomyolysis, a complete breakdown of muscle cells that can lead to kidney failure and death. Some experts believe that pravastatin (Pravachol®) and fluvastatin (Lescol®) may have less potential for this deadly drug interactions. The data at this time are not sufficient to declare one statin drug safer or more dangerous than the others.

Statin drugs also inhibit the intrinsic biosynthesis of Coenzyme Q10 (CoQ10), a central compound in the mitochondrial respiratory chain. However, CoQ10 is indispensable for optimum cardiac function and thus serious complications will occur unless one supplements CoQ10 back into the diet. A prescription for lipid-lowering statin drugs should always be accompanied with a recommendation to take CoQ10, because if a person is deficient in CoQ10, heart failure is more likely.

In addition, statin drugs have also been linked to various forms of cancer in laboratory animals. It will be years before the full side effects of statin drugs are completely understood.

Using statin drugs on a short-term basis to normalize blood cholesterol is a reasonable alternative if a drug-free approach fails. Anyone on long term statin type cholesterol lowering drug would be wise to get regular checkups, especially with respect to liver function.

Fibrates

Fibrates, or fibric acid derivatives, are mainly used for lowering triglycerides and increasing HDL cholesterol. They work by increasing the activity of an enzyme that breaks down some kinds of cholesterol helping the liver to eliminate cholesterol and blocking the production of triglycerides and cholesterol. Fibrates can significantly reduce triglyceride levels and can increase HDL cholesterol, but they don't lower LDL cholesterol levels as effectively. The currently available fibrates are bezafibrate (Bezalip®), fenofibrate (Lipidil®) and gemfibrozil (Lopid®). Their most common side effects include nausea, stomach pain, gas, diarrhea, constipation, itching, rash, tiredness and muscle aches.

Resins

Resins, or bile acid sequestrants, are primarily used to lower total cholesterol and LDL cholesterol. They work by making the liver use more of its own cholesterol, which lowers the amount of cholesterol in the blood. The two currently available resins, cholestyramine (Questran®) and colestipol (Colestid®), can reduce LDL cholesterol levels but tend to raise triglyceride levels slightly. Side effects may include constipation, stomach pain, gas, heartburn, nausea, diarrhea and loss of appetite.

Nutritional Supplements

As an alternative to prescription medications, nutritional supplements may also be used to lower total blood cholesterol levels. Examples of nutritional supplements that appear to be effective are: Coenzyme Q10 (CoQ10); L-carnitine; garlic; digestive enzymes, such as lipase and amylase; probiotics or “friendly bacteria” such as L. Acidophilus; Milk Thistle (Silybum marianum); herb tea; pantethine and pantothenic acid; and policosanol.

A mixture of high purity, high molecular weight straight chain aliphatic alcohols (collectively referred to herein as policosanol) has garnered much interest in recent years as a natural supplement for its cholesterol-lowering effects (Gouni-Berthold I., et al., Am. Heart J, 143(2):356-365 (2002)). The main constituents of policosanol are tetracosanol, hexacosanol, octacosanol, and triacontanol, while eicosanol, docosanol, heptacosanol, nonacosanol, dotriacontanol, tetratriacontanol, and hexatriacontanol make up the remaining minor constituents of the straight chain aliphatic alcohols. There is a significant body of evidence demonstrating the benefits of policosanol with respect to cardiovascular disease. In the mid to late nineties, one research group proposed that policosanol was able to reduce endothelial damage by inhibiting the production of foam cells (Noa, M., et al., J. Pharm. Pharmacol., 48(3):306-309 (1996); Noa, M., et al., J. Pharm. Pharmacol., 49(10):999-1002 (1997)). Foam cells are macrophages that can migrate into the endothelium of the blood vessels and contribute to atherosclerotic plaque formation (Physicians' Desk Reference, 50 ed. Montvale, N.J.: Medical Economics Company; 2002)). Other researchers believe policosanol has a modulating effect on HMG-CoA reductase, the rate-controlling enzyme in cholesterol biosynthesis, but the precise mechanism remains unclear (Menendez, R., et al., Biol. Res., 27(3-4):199-203 (1994); Menendez, R., et al., Biol. Res., 29(2):253-257 (1996); and Menendez, R., et al., Arch. Med. Res., 32(1):8-12 (2001)). Still, other investigators believe policosanol may inhibit cholesterol synthesis in the liver at a step before mevalonate production, but total inhibition of the HMG-CoA reductase is doubtful (Gouni-Berthold, I., et al., Am. Heart J, 143(2):356-365 (2002)). More recent work suggests policosanol inhibits LDL cholesterol oxidation (Menendez, R., et al., Can. J. Physiol. Pharmacol., 80(1):13-21 (2002); Menendez, R., et al., Br. J. Clin. Pharmacol., 50(3):255-262 (2000)). This was revealed when markers of peroxidation, such as thiobarbituric acid reactive substances (TBARS), and malondialdehyde (MDA) were lower in the cultures treated with policosanol. Oxidation of LDL cholesterol has been linked to heart disease and was the recent cover story in Scientific American magazine (Physicians' Desk Reference, 50 ed., Montvale, N.J.: Medical Economics Company; 2002)). Bi-products of LDL oxidation are bioactive, and secrete inflammatory cytokines, growth factors and cell surface adhesion molecules. In response to these oxidative bi-products, smooth muscle cells proliferate in the wall of the artery, resulting in the narrowing of the lumen and eventual blockage. Oxidized LDL cholesterol can also inhibit the production of prostacyclin and nitric oxide, which act as vasodilators and inhibitors of platelet aggregation.

While there are no known side effects related to the use of policosanol and the percentage decrease in the reduction of total cholesterol as well as total LDL-cholesterol is statistically significant, it is not as significant as the reduction that occurs as a result of administering the prescription medications discussed previously.

It would be advantageous to provide a unique policosanol-containing formulation which allows individuals to significantly lower and maintain healthy cholesterol levels in the blood, as is accomplished with prescription medications, while not exposing the individual to the same deleterious side effect that result from long term use of prescription medications.

### SUMMARY OF THE INVENTION

The present invention provides a therapeutic composition for reducing serum cholesterol and LDL-cholesterol levels in humans and animals, and a method for reducing serum cholesterol and LDL-cholesterol levels in humans and animals by administering the composition to a human or animal in need thereof. The composition comprises a mixture of high purity, high molecular weight straight chain primary aliphatic alcohols and 3-hydroxy-3-methylglutaryl co-enzyme A (HMG-CoA) reductase inhibitor. Wherein the composition comprises from about 1% to about 90% by weight policosanol and from about 5% to about 75% by weight of HMG-CoA reductase inhibitor. The composition further comprises from 0% to about 65% by weight of pharmaceutically acceptable formulation aids, such as diluents, stabilizers, binders, buffers, lubricants, coating agents, preservatives, emulsifiers and suspension agents.

In one embodiment of this aspect of the invention, the policosanol comprises at least one high molecular weight straight chain primary aliphatic alcohol selected from 20 to 36 carbon atoms, and an HMG-CoA reductase inhibitor is selected from a class of statin drugs, and the composition is further characterized by a combination policosnaol and HMG-CoA reductase inhibitor in a quantitative ratio from 100:1 to 0.01:1 by weight. In yet another embodiment, the composition of the present invention comprises policosanol having the following quantitative composition:

| | Proportion in |
|---|---|
| Components | the mixture |
| 1-eicosanol (C₂₀) | 0-5% |
| 1-docosanol (C₂₂) | 0-5% |
| 1-tetracosanol (C₂₄) | 0-30% |
| 1-hexacosanol (C₂₆) | 5-30% |
| 1-heptacosanol (C₂₇) | 0-5% |
| 1-octacosanol (C₂₈) | 5-80% |
| 1-nonacosanol (C₂₉) | 0-5% |
| 1-triacontanol (C₃₀) | 5-40% |
| 1-dotriacontanol (C₃₂) | 1-25% |
| 1-tetratriacontanol (C₃₄) | 0-7% |
| 1-hexatriacontanol (C₃₆) | 0-5% |

and an HMG-CoA reductase inhibitor which may be selected from the group consisting of lovastatin, fluvastatin, rosuvastatin, pravastatin, simvastatin, cervastatin, and atorvastatin; and the composition is further characterized by a combination of policosanol and HMG-CoA reductase inhibitor in a quantitative ratio from 3:1 to 0.10:1 by weight.

In still another aspect, the present invention relates to a method for treating or preventing hypercholesterolemia related diseases which comprises administering a pharmaceutically effective amount of a composition comprising policosanol and HMG-CoA reductase inhibitor to a mammal, e.g., a human.

In yet another aspect, the present invention relates to a method for reducing total cholesterol and LDL-cholesterol levels which comprises administering a pharmaceutically effective amount of a composition comprising policosanol and HMG-CoA reductase inhibitor to a mammal, e.g., a human, in need thereof.

In still yet another aspect, the present invention relates to a method of using a composition comprising policosanol and HMG-CoA reductase inhibitor which comprises administering said composition to reduce and/or prevent hypercholesterolemic diseases, total cholesterol, LDL-cholesterol, coronary heart disease (heart attacks and strokes), inflammation, immunoregulatory diseases, cardiovascular diseases, and/or neurodegenerative disorders in an individual in need thereof. The daily dosage is established between 1 to 100 mg of policosanol (preferably 3 to 20 mg) and 0.3 to 80 mg of HMG-CoA reductase inhibitor per day and is intended for ingestion in any type or form of foodstuff, capsule, tablet or liquid form.

The present invention further contemplates providing kits having one or more containers comprising the therapeutic composition of the present invention and a suitable excipient as described herein and a set of instructions, generally written instructions, although electronic storage media (e.g., magnetic diskette or optical disk) containing instructions are also acceptable, relating to the use and dosage of the therapeutic composition of the present invention for the intended treatment. The instructions included with the kit generally include information as to dosage, dosing schedule, and route of administration for the intended treatment. The containers of the therapeutic composition of the present invention may be unit doses, bulk packages (e.g., multi-dose packages) or sub-unit doses.

### DETAILED DESCRIPTION OF THE INVENTION

The composition of the present invention comprises a mixture of high purity, high molecular weight straight chain primary aliphatic alcohols (referred collectively herein to as policosanol) and a 3-hydroxy-3-methylglutaryl co-enzyme A (HMG-CoA) reductase inhibitor (referred to herein as statin) as the primary therapeutic agents to be administered for the purpose of reducing and/or preventing hypercholesterolemic diseases, total cholesterol, LDL-cholesterol, coronary heart disease (heart attacks and strokes), inflammation, immunoregulatory diseases, cardiovascular diseases, and/or neurodegenerative disorders in an individual in need thereof.

The therapeutic compositions of the present invention comprise statin and policosanol. The policosanol used in the present invention can be derived from any suitable source, each source being associated with a policosanol of particular characteristics, usually in terms of the relative proportions of its primary aliphatic alcohol components. For example, policosanol may be extracted and purifyied from a wide array of starting materials, such as but not limited to, natural waxes, such as but not limited to, beeswax, carnauba wax, and candellia wax; bee pollen; oils, such as but not limited to, peanut oil, sesame oil, cod liver oil, rice bran oil, oat oil, and rosemary needles oil; and powders, such as but not limited to rice bran, containing primarily natural esters of aliphatic alcohols with caboxylic acids. Consequently, the quantitative compositions of policosanol can vary depending on the extraction process and starting materials that are used in its production. In general, it is possible to obtain policosanol having the quantitative composition shown in Table I.

TABLE I

| | Proportion in |
|---|---|
| Components | the mixture |
| 1-eicosanol (C₂₀) | 0-5% |
| 1-docosanol (C₂₂) | 0-5% |
| 1-tetracosanol (C₂₄) | 0-30% |
| 1-hexacosanol (C₂₆) | 5-30% |
| 1-heptacosanol (C₂₇) | 0-5% |
| 1-octacosanol (C₂₈) | 5-80% |
| 1-nonacosanol (C₂₉) | 0-5% |
| 1-triacontanol (C₃₀) | 5-40% |
| 1-dotriacontanol (C₃₂) | 1-25% |
| 1-tetratriacontanol (C₃₄) | 0-7% |
| 1-hexatriacontanol (C₃₆) | 0-5% |

U.S. Pat. Nos. 5,663,156; 5,856,316; 6,197,832; 6,225,354; and 6,596,776, all of which are incorporated herein by reference, disclose policosanol compositions that are specific to the starting material and extraction processes used. It should be noted that while any commercially available policosanol or any of the policosanols disclosed in the above-referenced patents are suitable for use in the present invention, for purposes of the remainder of this discussion the policosanol and methodologies disclosed in U.S. Pat. No. 6,596,776 will be referenced. Specifically, the policosanol used in the present invention is obtained from beeswax and has the formulation set forth in Table II.

TABLE II

| | Proportion in |
|---|---|
| Components | the mixture |
| 1-eicosanol (C₂₀) | 0-5% |
| 1-docosanol (C₂₂) | 0-5% |
| 1-tetracosanol (C₂₄) | 13-28% |
| 1-hexacosanol (C₂₆) | 5-30% |
| 1-heptacosanol (C₂₇) | 0-5% |
| 1-octacosanol (C₂₈) | 15-25% |
| 1-triacontanol (C₃₀) | 25-40% |
| 1-dotriacontanol (C₃₂) | 5-15% |
| 1-tetratriacontanol (C₃₄) | 0-5% |

The process used to isolate the policosanol, described in Table II above, is incorporated herein by reference, and is briefly described as follows. Beeswax is initially subjected to a homogenous phase saponification step after which the saponified beeswax is dried and ground to a particle mesh size of 100-500 microns. Alternatively, unsaponified beeswax, of varying purity, may be used as the starting material and is initially dried and ground to a particle mesh size of 100-200 microns. The particles of saponified or unsaponified beeswax are placed into a conventional solid-liquid extractor and a hot organic solvent is introduced and contacted with the beeswax particles. The suspension is mixed and then hot-filtered to remove any solids.

The resulting extract is then maintained within the temperature range of 2° C.-10° C. causing the aliphatic alcohols to solidify and form a suspension. The suspension is filtered and the first solids are recovered and air dried. The dried solids obtained after drying are then sent to a purifier where they are contacted with and dissolved in a second hot solvent and hot-filtered. This solution is then cooled and the second solids collected and dried by vacuum. The dried solids obtained from the second purification step are contacted with another hot organic solvent, which dissolves the solids. This solution is hot-filtered and chilled, and the third solids collected, dried, and powdered to become the final product disclosed in Table II.

After the particles are dried, they are then ready to be combined with niacin and/or thereby forming the therapeutic composition of the present invention which can be formulated into a conventional pharmaceutical formulation such as tablets, capsules, etc., for administration.

As discussed previously, statin and policosanol lower serum cholesterol by two independent mechanisms of action. However, the inventor believes that both compounds together will have a synergistic effect on lowering serum cholesterol. As previously mentioned, the mode of action of the statin drugs is to inhibit HMG-CoA reductase. HMG-CoA reductase is the enzyme responsible for the conversion of HMG-CoA to mevalonate, an early step in cholesterol biosynthesis. Policosanol on the other hand decreases cholesterol synthesis from acetate, not from mevalonate, suggesting an inhibition of 3-hydroxy-3-methylglutaryl co-enzyme A (HMG-CoA) reductase. However, unlike statin drugs, policosanol does not competiteively or non-competitively inhibit HMG-CoA reductase, suggesting that policosanol interferes with the synthesis or degredation of the enzyme (Menendez, R., et al., Biol. Res., 29:253-257 (1996); Menendez, R., et al., Arch. Med. Res., 32:8-12 (2001); and Menendez, R., et al., Biol. Res., 27:199-203 (1994)). Furthermore, one of the harmful side effects of the statins is that they also inhibit the intrinsic biosynthesis of Coenzyme Q10 (CoQ10), a central compound in the mitochondrial respiratory chain. CoQ10 is indispensable for optimum cardiac function, and serious complications will occur unless one supplements coenzyme Q10 back into the diet. In contrast, policosanol does not interfere with CoQ10. Thus, the combination of both policosanol and statin into a single composition provides a more effective treatment for elevated serum cholesterol than would be expected from the additive effect of both components. Furthermore, the composition of the present invention will contain a significantly lower amount of the recommended daily dosage of the statin constituent thus decreasing the side effects associated with the use of statins while simultaneously achieving an effective treatment for elevate srum cholesterol and LDL-cholesterol.

The statins currently available for use in the present invention and the associated recommended daily dosage include Mevacor® (lovastatin, 20-80 mg/day), Lescol® (fluvastatin, 20-80 mg/day), Crestor® (rosuvastatin, 5-10 mg/day), Pravachol® (pravastatin, 20-40 mg/day), Zocor® (simvastatin, 5-80 mg/day), Baycol® (cervastatin, 0.3-0.8 mg/day), and Lipitor® (atorvastatin, 10-80 mg/day). The dose of these medicines will be variable for different patients and dose levels can be determined as is normally employed in the art, for example, as indicated in the Physician's Desk Reference and The Merck Index (Twelfth Edition), the contents of both of which are incorporated herein by reference.

Policosanol/HMG-CoA Reductase Inhibitor Formulations of the Present Invention

The formulations of the present invention comprise compositions made by combining policosanol with a statin. Such compositions can comprise policosanol with a statin in a quantitative ratio from about 100:1 to about 0.01:1 by weight, to from about 10:1 to about 0.10:1 by weight, e.g., from about 3:1 to about 0.33:1 by weight, typically from about 2:1 to about 0.5:1. Compositions of the present invention may further contain 1:1 weight ratios of policosanol with statin.

Policosanol is extremely well tolerated. In animal toxicity studies doses up to 500 mg/kg/day, a dose that is 1500 times the normal human dose of 20 mg/day have shown no negative effects with respect to carcinogenesis, reproduction, growth, and development. Total doses of policosanol according to the present invention range from 1 mg to 100 mg per day, in another embodiment it is contemplated that 5 mg to 40 mg per day is used and in yet another embodiment it is contemplated that the dose would be in the range of 10 to 20 mg per day.

A wide variety of statin preparations are available from different manufacturers, each having unique bioavailability, pharmokinetic, and safety profiles. In general, lower doses of statins are contemplated for use, because they maintain beneficial lipid effects while minimizing adverse side affects the total dose for this component of the composition of the present invention can range from about 0.1 mg/day to about 160 mg/day, or any other dose, depending upon the specific statin employed.

According to the present invention, one of the synergistic effects of the active compounds that make up the composition of the present invention is the ability to achieve the same end results that can possibly be achieved with the use of the statin alone while significantly decreasing the side effects associated with the use of statins. Consequently, while the recommended daily dosage of each statin can be combined with policosanol to form the composition of the present invention, it is preferable to use a smaller dose than recommended. With respect to lovastatin it is contemplated that a useful dose is in the range of 1-160 mg/day, in another embodiment the useful dose is in the range of 5-60 mg/day and in yet another embodiment the useful dose is in the range of 10-40 mg/day; the useful dose for fluvastatin is contemplated to be in the range of 1-160 mg/day, in another embdiment the useful dose is in the range of 5-60 mg/day, and in yet another embodiment the useful dose is in the range of 10-40 mg/day. The useful dose for rosuvastatin is contemplated to be in the rnage of 1-20 mg/day, in another embodiment the useful dose is in the range of 2-8 mg/day, and in yet another embodiment the useful dose is in the range of 4-6 mg/day. The useful dose for pravastatin is contemplated to be in the range of 1-80 mg/day, in another embodiment the useful dose is in the range of 5-30 mg/day, and in yet another embodiment the useful dose is in the range of 10-20 mg/day. The useful dose for simvastatin is contemplated to be in the range of 1-160 mg/day, in another embodiment the useful dose is in the range of 5-60 mg/day and in yet another embodiment the useful dose is in the range of 10-40 mg/day. The useful dose for cervastatin is contemplated to be 0.1-1.6 mg/day, in another embodiment the useful dose is 0.1-0.6 mg/day and in yet another embodiment the useful dose is in the range of 0.1-0.4 mg/day. The useful dose for atorvastatin is contemplated to be in the range of 1-160 mg/day, in another embodiment the useful dose is in the range of 5-60 mg/day and in yet another embodiment the useful dose is in the range of 10-40 mg/day. Other dosing ranges may be further determined by one skilled in the art as indicated in the Physician's Desk Reference and The Merck Index (Twelfth Edition).

The compositions of the present invention can be taken in amounts sufficient to provide the desired dosages discussed above. The formulation can be taken one or more times a day.

The pharmaceutical formulations of the present invention can contain as active ingredients from about 0.5 to about 95.0 wt % of policosanol and statin. This dosage is obtained by mixing the policosanol and statin with different excipients such as agglutinants, disintegrators, lubricants, sliders or just fillers. These excipients include lactose, corn starch, saccharose, magnesium stearate, microcrystalline cellulose, sodium croscarmellose gelatin, cellulose acetophtalate, titanium dioxide, special talc for tablets and polyethylene glycol.

The pharmaceutical composition, of the present invention may be administered to humans and animals. The daily dosage of this compostion to be used for the reduction and/or prevention of hyper-cholesterolemic diseases, cholesterol, LDL-cholesterol, coronary heart disease (heart attacks and stroke), inflammation, deep-vein thrombosis or immunoregulatory diseases, cardiovascular disease and neurodegenerative disorders is established between 1 to 100 mg/day for the policosanol component and 0.1 to 160 mg/day for the statin component depending on which statin is present and is intended for administration in a variety of ways discussed in further detail below.

As will be appreciated by one knowledgeable in the art, the therapeutic composition of the present invention may be combined or used in combination with other treatments known in the art.

The therapeutic composition of the present invention may be packaged in any convenient, appropriate packaging.

The compositions of the invention may be in a form suitable for oral use (for example, as tablets, lozenges, hard or soft capsules, aqueous or oily suspensions, emulsions, dispersible powders or granules, syrups or elixirs), for topical use (for example, as creams, ointments, gels, or aqueous or oily solutions or suspensions), for administration by inhalation (for example, as a finely divided powder or a liquid aerosol), for administration by insufflation (for example, as a finely divided powder) or for parenteral administration (for example, as a sterile aqueous or oily solution for intravenous, subcutaneous, or intramuscular dosing or as a suppository for rectal dosing). For example, compositions intended for oral use may contain, one or more coloring, sweetening, flavoring and/or preservative agents.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents, such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example, sorbitan monooleate) and condensation products of partial esters with ethylene oxide, such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedures well known in the art.

Compositions for administration by insufflation may be in the form of a finely divided powder containing particles of average diameter of, for example, 30 μm or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulations, see Chapter 25.2 in Volume 5 of Comprehensive Medicinal Chemistry (Corwin Hansch; Chairman of Editorial Board), Pergamon Press 1990, which is specifically incorporated herein by reference.

The amount of the active ingredients comprising the composition of this invention that is combined with one or more excipients to produce a single dosage form will necessarily vary depending upon the host treated and the particular route of administration. For example, a formulation intended for oral administration to humans may contain, for example, from 0.5-30 g of active agent compounded with an appropriate and convenient amount of excipients which may vary from about 5 to about 95 percent by weight of the total composition. Dosage unit forms will generally contain about 0.5-30 g of an active ingredient. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

In order to use the formulation of policosanol and statin and/or for the therapeutic treatment (including prophylactic treatment) of mammals including humans according to the methods of this invention, it is normally formulated in accordance with standard pharmaceutical practice as a pharmaceutical composition as discussed above. According to this aspect of the invention there is provided a pharmaceutical composition comprising policosanol and statin and/or in association with a pharmaceutically acceptable diluent or carrier, wherein the policosanol and statin and/or are present in an amount for effectively treating or preventing hypercholesterolemic diseases, total cholesterol, LDL-cholesterol, LDL/HDL ratio, Lp(a), triglycerides, coronary heart disease (heart attacks and strokes), inflammation, immunoregulatory diseases, cardiovascular diseases, deep-vein thrombosis, anxiety, depression and/or neurodegenerative disorders, and/or raise HDL cholesterol, in an individual in need thereof.

The composition of the present invention can be administered to a patient by any available and effective delivery system including, but not limited to, parenteral, transdermal, intranasal, sublingual, transmucosal, intra-arterial, or intradermal modes of administration in dosage unit formulations containing conventional nontoxic pharmaceutically acceptable carriers, adjuvants, and vehicles as desired, such as a depot or a controlled release formulation.

For example, a pharmaceutically acceptable formulation of the composition of the present invention may be formulated for parenteral administration, e.g., for intravenous, subcutaneous, or intramuscular injection. For an injectable formulation, a dose of the composition of the present invention may be combined with a sterile aqueous solution which is preferably isotonic with the blood of the patient. Such a formulation may be prepared by dissolving a solid active ingredient in water containing physiologically-compatible substances such as sodium chloride, glycine, and the like, and having a buffered pH compatible with physiological conditions so as to produce an aqueous solution, and then rendering the solution sterile by methods known in the art. The formulations may be present in unit or multi-dose containers, such as sealed ampules or vials. The formulation may be delivered by any mode of injection, including, without limitation, epifascial, intracutaneous, intramuscular, intravascular, intravenous, parenchymatous, subcutaneous, oral or nasal preparations (see, for example, U.S. Pat. No. 5,958,877, which is specifically incorporated herein by reference).

Controlled/Extended/Sustained/Prolonged Release Administration

Another aspect of this invention provides methods of treating hypercholesterolemic diseases, total cholesterol, LDL-cholesterol, coronary heart disease (heart attacks and strokes), inflammation, immunoregulatory diseases, deep-vein thrombosis, cardiovascular diseases, anxiety, depression and/or neurodegenerative disorders by delivering the composition of the present invention to a patient as a controlled release formulation. As used herein, the terms “controlled,” “extended,” “sustained,” or “prolonged” release of the composition of the present invention will collectively be referred to herein as “controlled release,” and includes continuous or discontinuous, and linear or non-linear release of the composition of the present invention. There are many advantages for a controlled release formulation of the composition of the present invention. Among these are to effectively suppress cholesterol synthesis during a period when the patient is not readily able or willing to periodically ingest the composition of the present invention. The composition of the present invention is preferably administered following the evening meal and prior to bedtime in a single dose. The single dose of composition of the present invention preferably is administered by ingestion of one or more controlled release unit dosage forms so that effective statin and/or and policosanol levels are maintained throughout the night, i.e., during the peak periods of serum lipid/lipid component biosynthesis.

1. Tablets

A controlled release tablet suitable for purposes of this invention is disclosed in U.S. Pat. No. 5,126,145, which is incorporated by reference herein. This tablet comprises, in admixture, about 5-30% high viscosity hydroxypropyl methyl cellulose, about 2-15% of a water-soluble pharmaceutical binder, about 2-20% of a hydrophobic component such as a waxy material, e.g., a fatty acid, and about 30-90% active ingredient.

More specifically, one such controlled release tablet of this invention comprises: (a) about 5-20 percent by weight hydroxypropyl methylcellulose having a viscosity of about 10,000 CPS or greater, a substitution rate for the methoxyl group of about 7-30% and a substitution rate for the hydroxypropoxyl group of about 7-20%; (b) about 2-8 percent hydroxypropyl methylcellulose having a viscosity of less than about 100, CPS methyl cellulose, or polyvinyl pyrollidone; (c) about 5-15 percent by weight hydrogenated vegetable oil or stearic acid; and (d) about 30-90% of a pharamaceutical composition of this invention.

High viscosity water-soluble 2-hydroxypropyl methyl cellulose (HPMC) is particularly preferred for use in the present tablets and in the controlled-release tablet coating due to its sustaining properties with respect to policosanol and statin release. “Water soluble” for purposes of this application means that two grams of powdered cellulose ether can be dispersed by stirring into 100 grams of water at a temperature between 0° C.-100° C. to provide a substantially clear, stable aqueous composition or dispersion (when the dispersion is brought to 20° C.). A particularly preferred high viscosity HMPC ((Methocel® K100MCR) has a nominal viscosity, two percent solution, of about 100,000 CPS, methoxyl content of about 19-24, a hydroxypropyl content of about 7-12 percent, and a particle size where at least 90% passes through a USS 100 mesh screen. Low viscosity HPMC is preferred as the binder component of the tablet. A particularly preferred low viscosity HPMC (Methocel® EIS) has a methoxyl content of about 20-30%, a hydroxylpropyl content of about 7-12 percent, and a particle size where 100% will pass through a USS No. 30 mesh screen and 99% will pass through a USS 40 mesh screen. In some cases, a portion of the high viscosity HPMC can be replaced by a medium viscosity HPMC, i.e., of about 2000-8,000 cps.

The viscosities reported herein are measured in centipoises (cps or cP), as measured in a 2% by weight aqueous solution of the cellulose either at 20° C. using a rotational viscometer. A “high viscosity” cellulose ether possesses a viscosity of at least about 10,000 cps i.e., about 50,000-100,000 cps. A low-viscosity cellulose ether possesses a viscosity of less than about 100 cps, i.e., about 10-100 cps.

Useful hydrophobic components include natural and synthetic waxes such as beeswax, carnauba wax, paraffin, spermaceti, as well as synthetic waxes, hydrogenated vegetable oils, fatty acids, fatty alcohols and the like.

The controlled release policosanol and statin tablets preferably can be formulated to contain 10 mg, 20 mg or 40 mg of policosanol and 0.5-30g of statin depending on the particular statin used, and are ingested orally.

Preferably, these tablets will release about 10-35 wt % of the total policosanol and statin within about 2 hours in an in vitro dissolution test, and about 40-70 wt % of the total policosanol and statin in eight hours.

These controlled released tablets can also be coated so as to further prolong the release of the statin into the gastrointestinal tract, or to prevent its release into the stomach, in order to prevent or attenuate the gastrointestinal side effects which can accompany statin administration.

For example, coatings comprising a major portion of a polymeric material having a high degree of swelling on contact with water or other aqueous liquids can be used to further prolong the release of the statin from the tablets core. Such polymers include, inter alia, cross-linked sodium carboxymethylcellulose (Acdisol-FMC), cross-linked hydroxypropylcellulose, hydroxymethylpropylcellulose, e.g., Methocel® K15M, Dow Chemical Co., carboxymethylamide, potassium methylacrylate divinylbenzene copolymer, polymethyl methacrylate, cross-linked polyvinylpyrrolidine, high molecular weight polyvinylalcohol, and the like. Hydroxypropylmethyl cellulose is available in a variety of molecular weights/viscosity grades from Dow Chemical Co. under the trademark Methocel®. See also, Alderman (U.S. Pat. No. 4,704,285). These polymers may be dissolved in suitable volatile solvents, along with dyes, lubricants, flavorings and the like, and coated onto the prolonged release tablets, e.g., in amounts equal to 0.1-5% of the total tablet weight, by methods well known to the art. For example, see Remington's Pharmaceutical Sciences, A. Osol, ed., Mack Publishing Co., Easton, Pa. (16th ed. 1980) at pages 1585-1593.

Enteric coatings can also be provided to the prolonged release tablets to prevent release of the statin until the tablet reaches the intestinal tract. Such coatings comprise mixtures of fats and fatty acids, shellac and shellac derivatives and the cellulose acid phthlates, e.g., those having a free carboxyl consent of 9-15%. See, Remington's at page 1590, and Zeitova et al. (U.S. Pat. No. 4,432,966), for descriptions of suitable enteric coating compositions.

2. Films

This invention further provides a prophylaxis for or method of treating a patient following an invasive cardiac procedure comprising administering biodegradable, biocompatible polymeric film comprising statin and policosanol to a patient. The polymeric films are thin compared to their length and breadth. The films typically have a uniform selected thickness between about 60 micrometers and about 5 mm. Films of between about 600 micrometers and 1 mm and between about 1 mm and about 5 mm thick, as well as films between about 60 micrometers and about 1000 micrometers, and between about 60 and about 300 micrometers are useful in the manufacture of therapeutic implants for insertion into a patient's body. The films can be administered to the patient in a manner similar to methods used in adhesion surgeries. For example, a policosanol and statin film formulation can be sprayed or dropped onto a cardiac tissue site or artery during surgery, or a formed film can be placed over the selected tissue site. In an alternative embodiment, the film can be used as controlled release coating on a medical device such as a stent, as is discussed in further detail below.

Either biodegradable or nonbiodegradable polymers may be used to fabricate implants in which the statin and policosanol is uniformly distributed throughout the polymer matrix. A number of suitable biodegradable polymers for use in making the biodegradable films of this invention are known to the art, including polyanhydrides and aliphatic polyesters, preferably polylactic acid (PLA), polyglycolic acid (PGA) and mixtures and copolymers thereof, more preferably 50:50 copolymers of PLA:PGA and most preferably 75:25 copolymers of PLA:PGA. Single enantiomers of PLA may also be used, preferably L-PLA, either alone or in combination with PGA. Polycarbonates, polyfumarates and caprolactones may also be used to make the implants of this invention.

A plasticizer may be incorporated in the biodegradable film to make it softer and more pliable for applications where direct contact with a contoured surface is desired.

The polymeric films of this invention can be formed and used as flat sheets, or can be formed into three-dimensional conformations or “shells” molded to fit the contours of the tissue site into which the film is inserted.

To make the polymeric films of this invention, a suitable polymeric material is selected, depending on the degradation time desired for the film. Selection of such polymeric materials is known to the art. A lower molecular weight, e.g., around 20,000 daltons, 50:50 or 55:45 PLA:PGA copolymer is used when a shorter degradation time is desired. To ensure a selected degradation time, the molecular weights and compositions may be varied as known to the art.

Polymeric films of this invention may be made by dissolving the selected polymeric material in a solvent known to the art, e.g., acetone, chloroform or methylene chloride, using about 20 mL solvent per gram of polymer. The solution is then degassed, preferably under gentle vacuum to remove dissolved air and poured onto a surface, preferably a flat non-stick surface such as BYTAC (Trademark of Norton Performance Plastics, Akron, Ohio) non-stick coated adhesive-backed aluminum foil, glass or TEFLON™ non-stick polymer. The solution is then dried, preferably air-dried, until it is no longer tacky and the liquid appears to be gone. The known density of the polymer may be used to back-calculate the volume of solution needed to produce a film of the desired thickness.

Films may also be made by heat pressing and melt forming/drawing methods known to the art. For example, thicker films can be pressed to form thinner films, and can be drawn out after heating and pulled over forms of the desired shapes, or pulled against a mold by vacuum pressure.

The amount of the composition of the present invention to be incorporated into the polymeric films of this invention is an amount effective to show a measurable effect in treating hypercholesterolemia. The composition of the present invention can be incorporated into the film by various techniques such as by solution methods, suspension methods, or melt pressing.

Solid implants comprising the composition of the present invention can also be made into various shapes other than films by injection molding or extrusion techniques. For example, the implant can comprise a core material such as ethylene/vinyl acetate copolymer, and a vinyl acetate content of 20% by weight or more and which functions as a matrix for the composition of the present invention, in a quantity which is sufficient for a controlled release of the composition of the present invention, and a membrane which encases the core material and also consists of EVA material and an acetate content of less than 20% by weight. The implant can be obtained, for example, by means of a co-axial extrusion process, a method in which the two EVA polymers are extruded co-axially with the aid of a co-axial extrusion head. The co-axial extrusion process is art known per se so that it will not be gone into further within the scope of this description.

3. Transdermal Patch Device

Transdermal delivery involves delivery of a therapeutic agent through the skin for distribution within the body by circulation of the blood. Transdermal delivery can be compared to continuous, controlled intravenous delivery of a drug using the skin as a port of entry instead of an intravenous needle. The therapeutic agent passes through the outer layers of the skin, diffuses into the capillaries or tiny blood vessels in the skin and then is transported into the main circulatory system.

Transdermal patch devices which provide a controlled, continuous administration of a therapeutic agent through the skin are well known in the art. Such devices, for example, are disclosed in U.S. Pat. Nos. 4,627,429; 4,784,857; 5,662,925; 5,788,983; and 6,113,940, which are all incorporated herein by reference. Characteristically, these devices contain a drug impermeable backing layer which defines the outer surface of the device and a permeable skin attaching membrane, such as an adhesive layer, sealed to the barrier layer in such a way as to create a reservoir between them in which the therapeutic agent is placed. In one embodiment of the present invention a formulation of the composition of the present invention is introduced into the reservoir of a transdermal patch.

4. Medical Devices

Another embodiment contemplates the incorporation of the composition of the present invention into a medical device that is then positioned to a desired target location within the body, whereupon the composition of the present invention elutes from the medical device. As used herein, “medical device” refers to a device that is introduced temporarily or permanently into a mammal for the prophylaxis or therapy of a medical condition. These devices include any that are introduced subcutaneously, percutaneously or surgically to rest within an organ, tissue or lumen. Medical devices include, but are not limited to, stents, synthetic grafts, artificial heart valves, artificial hearts and fixtures to connect the prosthetic organ to the vascular circulation, venous valves, abdominal aortic aneurysm (AAA) grafts, inferior venal caval filters, catheters including permanent drug infusion catheters, embolic coils, embolic materials used in vascular embolization (e.g., PVA foams), mesh repair materials, a Dracon vascular particle orthopedic metallic plates, rods and screws and vascular sutures.

The devices of this invention provide a therapeutically effective amount of the composition of the present invention to a targeted site such as a diseased or injured bodily tissue or organ such as by elution. The precise desired therapeutic effect will vary according to the condition to be treated, the formulation to be administered, and a variety of other factors that are appreciated by those of ordinary skill in the art. The amount of the composition of the present invention needed to practice the claimed invention also varies with the nature of the device used. For purposes of this invention, “elution” refers to any process of release that involves extraction or release by direct contact of the coating with bodily fluids.

In one embodiment, the medical device to be coated with the composition of the present invention is a stent or catheter for performing or facilitating a medical procedure. Accordingly, the present invention may be used in conjunction with any suitable or desired set of stent components and accessories, and it encompasses any of a multitude of stent designs. These stent designs may include for example a basic solid or tubular flexible stent member or a balloon catheter stent, up to complex devices including multiple tubes or multiple extruded lumens, as well as various accessories such as guide wires, probes, ultrasound, optic fiber, electrophysiology, blood pressure or chemical sampling components. In other words, the present invention may be used in conjunction with any suitable stent or catheter design, and is not limited to a particular type of catheter.

In another embodiment, the medical device can be designed to have pores for the delivery of the composition of the present invention to the desired bodily location, and can be prepared by the method disclosed in U.S. Pat. No. 5,972,027, which is incorporated herein by reference. Briefly, the method comprises providing a powdered metal or polymeric material, subjecting the powder to high pressure to form a compact, sintering the compact to form a final porous metal or polymer, forming a stent from the porous metal and, optionally, loading at least the composition of the present invention (and optionally one or more additional drugs) into the pores. For example, the stent may be impregnated with the composition of the present invention and optionally one or more additional drugs by any known process in the art, including high pressure loading in which the stent is placed in a bath of a composition of this invention and subjected to high pressure or, alternatively, subjected to a vacuum. The composition may be carried in a volatile or non-volatile solution. In the case of a volatile solution, following loading of the drug(s), the volatile carrier solution may be volatilized. In the case of the vacuum, the air in the pores of the metal stent is evacuated and replaced by the solution containing the comosition. Alternatively, rather than loading the porous stent with the comosition, the stent is instead implanted in the desired bodily location, and then the comosition is injected through a delivery tubing to the hollow stent and then out the pores in the stent to the desired location.

In another embodiment, the stent can be designed to contain reservoirs or channels which could be loaded with the composition of the present invention as described in U.S. Pat. No. 6,273,913 B1, which is incorporated herein by reference. A coating or membrane of biocompatible material could be applied over the reservoirs which would control the diffusion of the drug from the reservoirs to the artery wall. One advantage of this system is that the properties of the coating can be optimized for achieving superior biocompatibility and adhesion properties, without the additional requirement of being ale to load and release the drug. The size, shape, position, and number of reservoirs can be used to control the amount of drug, and therefore the dose delivered.

The stent can be made of virtually any biocompatible material having physical properties suitable for the design, and can be biodegradable or nonbiodegradable. The material can be either elastic or inelastic, depending upon the flexibility or elasticity of the polymer layers to be applied over it. Accordingly, the medical devices of this invention can be prepared in general from a variety of materials including ordinary metals, shape memory alloys, various plastics and polymers, carbons or carbon fibers, cellulose acetate, cellulose nitrate, silicone and the like.

For example, a medical device, such as but not limited to a stent, according to this invention can be composed of polymeric or metallic structural elements onto which a matrix is applied or the stent can be a composite of the matrix intermixed with a polymer.

Suitable biocompatible metals for fabricating the expandable stent include high grade stainless steel, titanium alloys including NiTi (a nickel-titanium based alloy referred to as Nitinol), cobalt alloys including cobalt-chromium-nickel alloys such as Elgiloy® and Phynox®, a Niobium-Titanium (NbTi) based alloy, tantalum, gold, and platinum-iridium.

Suitable nonmetallic biocompatible materials include, but are not limited to, polyamides, polyolefins (e.g., polypropylene, polyethylene etc.), nonabsorbable polyesters (i.e. polyethylene terephthalate), and bioabsorbable aliphatic polyesters (e.g., homopolymers and copolymers of lactic acid, glycolic acid, lactide, glycolide, para-dioxanone, trimethylene carbonate, ε-caprolactone, etc. and blends thereof).

a. Matrix

In one embodiment, the medical device such as a stent or graft is coated with a matrix. The matrix used to coat the stent or graft according to this invention may be prepared from a variety of materials. A primary requirement for the matrix is that it be sufficiently elastic and flexible to remain unruptured on the exposed surfaces of the stent or synthetic graft.

(1) Naturally Occurring Materials

The matrix may be selected from naturally occurring substances such as film-forming polymeric biomolecules that may be enzymatically degraded in the human body or are hydrolytically unstable in the human body such as fibrin, fibrinogen, heparin, collagen, elastin, and absorbable biocompatable polysaccharides such as chitosan, starch, fatty acids (and esters thereof), glucoso-glycans, hyaluronic acid, carbon, laminin, and cellulose.

(2) Synthetic Materials

In one embodiment, the matrix that is used to coat the stent or synthetic graft may be selected from any biocompatible polymeric material capable of holding the composition of the present invention. The polymer chosen must be a polymer that is biocompatible and minimizes irritation to the vessel wall when the stent is implanted. The polymer may be either a biostable or a bioabsorbable polymer depending on the desired rate of release or the desired degree of polymer stability.

Suitable materials for preparing a polymer matrix include, but are not limited to, polycarboxylic acids, cellulosic polymers, silicone adhesive, fibrin, gelatin, polyvinylpyrrolidone, maleic anhydride polymers, polyamides, polyvinyl alcohols, polyethylene glycols, polyethylene oxides, glycosaminoglycans, polysaccharides, polyesters, poly(amino acids)polyurethanes, segmented polyurethane-urea/heparin, silicons, polyorthoesters, polyanhydrides, polycarbonates, polypropylenes, poly-L-lactic acids, polyglycolic acids, polycaprolactones, polyhydroxybutyrate valerates, polyacrylamides, polyethers, polyalkylenes oxalates, polyamides, poly(iminocarbonates), polyoxaesters, polyamidoesters, polyoxaesters containing amido groups, polyphosphazenes, vinyl halide polymers, polyvinylidene halides, polyacrylonitrile, polyvinyl ketones, polyvinyl aromatics (e.g., polystyrene), etheylene-methyl methacrylate copolymers, acrylonitrile-styrene copolymers, ABS resins and ethylene-vinyl acetate copolymers; polyamides, such as Nylon 66 and polycaprolactam; alkyl resins; polycarbonates; polyoxymethylenes; polyimides; polyethers; epoxy resins, polyurethanes; rayon; rayon-triacetate, cellulose, cellulose acetate, cellulose acetate butyrate; cellophane; cellulose nitrate; cellulose propionate; cellulose ethers (i.e. carboxymethyl cellulose and hydoxyalkyl celluloses) and mixtures and copolymers thereof.

The polymers used for coatings are preferably film-forming polymers that have molecular weight high enough as to not be waxy or tacky. The polymers also preferably adhere to the stent and are not so readily deformable after deposition on the stent as to be able to be displaced by hemodynamic stresses. The polymers molecular weight are preferably high enough to provide sufficient toughness so that the polymers will not be rubbed off during handling or deployment of the stent and will not crack during expansion of the stent.

In one embodiment, the matrix coating can include a blend of a first co-polymer having a first, high release rate and a second co-polymer having a second, lower release rate relative to the first release rate as described in U.S. Pat. No. 6,569,195 B2, which is incorporated herein by reference. The first and second copolymers are preferably erodible or biodegradable. In one embodiment, the first copolymer is more hydrophilic than the second copolymer. For example, the first copolymer can include a polylactic acid/polyethylene oxide (PLA-PEO) copolymer and the second copolymer can include a polylactic acid/polycaprolactone (PLA-PCL) copolymer. Formation of PLA-PEO and PLA-PCL copolymers is well known to those skilled in the art. The relative amounts and dosage rates of the composition of the present invention delivered over time can be controlled by controlling the relative amounts of the faster releasing polymers relative to the slower releasing polymers. For higher initial release rates the proportion of faster releasing polymer can be increased relative to the slower releasing polymer. If most of the dosage is desired to be released over a long time period, most of the polymer can be the slower releasing polymer.

Alternatively, a top coating can be applied to delay release of the active ingredients, or could be used as the matrix for the delivery of a different pharmaceutically active material. For example, layering of coatings of fast and slow hydrolyzing copolymers can be used to stage release of the drug or to control release of different agents placed in different layers. Polymers with different solubilities in solvents can be used to build up different polymer layers that may be used to deliver different active ingredients or control the release profile of a drug. For example since ε-caprolactone-co-lactide elastomers are soluble in ethyl acetate and ε-caprolactone-co-glycolide elastomers are not soluble in ethyl acetate. A first layer of ε-caprolactone-co-glycolide elastomer containing a drug can be over coated with c-caprolactone-co-glycolide elastomer using a coating solution made with ethyl acetate as the solvent. As will be readily appreciated by those skilled, in the art numerous layering approaches can be used to provide the desired delivery of the composition of the present invention.

In one embodiment the coating is formulated by mixing the composition of the present invention and optionally one or more additional therapeutic agents with the coating polymers in a coating mixture. The composition of the present invention and the therapeutic agent may be present as a liquid, a finely divided solid, or any other appropriate physical form. Optionally, the mixture may include one or more additives, e.g., nontoxic auxiliary substances such as diluents, carriers, excipients, stabilizers or the like. Other suitable additives may be formulated with the polymer and the composition of the present invention and pharmaceutically active agent or compound. For example, hydrophilic polymers selected from the previously described lists of biocompatible film forming polymers may be added to a biocompatible hydrophobic coating to modify the release profile (or a hydrophobic polymer may be added to a hydrophilic coating to modify the release profile). One example would be adding a hydrophilic polymer selected from the group consisting of polyethylene oxide, polyvinyl pyrrolidone, polyethylene glycol, carboxylmethyl cellulose, hydroxymethyl cellulose and combination thereof to an aliphatic polyester coating to modify the release profile. Appropriate relative amounts can be determined by monitoring the in vitro and/or in vivo release profiles for the composition of the present invention and the therapeutic agents.

b. Biodegradable Matrix

In one embodiment, the matrix is a synthetic or naturally occurring biodegradable polymer such as aliphatic and hydroxy polymers of lactic acid, glycolic acid, mixed polymers and blends, polyhydroxybutyrates and polyhydroxy-valeriates and corresponding blends, or polydioxanon, modified starch, gelatin, modified cellulose, caprolactaine polymers, polyacrylic acid, polymethacrylic acid or derivatives thereof, which will not alter the structure or function of the medical device. Such biodegradable polymers will disintegrate in a controlled manner (depending on the characteristics of the carrier material and the thickness of the layer(s) thereof), with consequent slow release of the composition of the present invention incorporated therein, while in contact with blood or other body fluids. A discussion of biodegradable coatings is provided in U.S. Pat. No. 5,788,979, which is specifically incorporated herein by reference.

c. Application of the Matrix to the Medical Device

In accordance with one embodiment of the present invention, the composition of the present invention is applied as an integral part of a coating on at least the exterior surface of the stent. The solution is applied to the stent and the solvent is allowed to evaporate, thereby leaving on the stent surface a coating of the polymer and the therapeutic substance. Typically, the solution can be applied to the stent by any suitable means such as, for example, by immersion, spraying, or deposition by plasma or vapor deposition. In order to coat a medical device such as a stent, the stent is dipped or sprayed with a liquid solution of the matrix of moderate viscosity. After each layer is applied, the stent is dried before application of the next layer. In one embodiment, a thin, paint-like matrix coating does not exceed an overall thickness of 100 microns. Whether one chooses application by immersion or application by spraying depends principally on the viscosity and surface tension of the solution, however, it has been found that spraying in a fine spray such as that available from an airbrush will provide a coating with the greatest uniformity and will provide the greatest control over the amount of coating material to be applied to the stent. In either a coating applied by spraying or by immersion, multiple application steps are generally desirable to provide improved coating uniformity and improved control over the amount of therapeutic substance to be applied to the stent. The amount of the composition of the present invention to be included on the stent can be readily controlled by applying multiple thin coats of the solution while allowing it to dry between coats. The overall coating should be thin enough so that it will not significantly increase the profile of the stent for intravascular delivery by catheter. The adhesion of the coating and the rate at which the composition of the present invention is delivered can be controlled by the selection of an appropriate bioabsorbable or biostable polymer and by the ratio of composition of the present invention to polymer in the solution.

In order to provide the coated stent according to this embodiment, a solution which includes a solvent, a polymer dissolved in the solvent, the composition of the present invention dispersed in the solvent, and optionally a cross-linking agent, is first prepared. It is important to choose a solvent and polymer that are mutually compatible with the composition of the present invention. It is essential that the solvent is capable of placing the polymer into solution at the concentration desired in the solution. It is also essential that the solvent and polymer chosen do not chemically alter the therapeutic character of the composition of the present invention. However, the composition of the present invention only needs to be dispersed throughout the solvent so that it may be either in a true solution with the solvent or dispersed in fine particles in the solvent. Preferable conditions for the coating application are when the polymer and composition of the present invention have a common solvent. This provides a wet coating that is a true solution. Less desirable, yet still usable are coatings that contain the composition of the present invention as a solid dispersion in a solution of the polymer in solvent. Under the dispersion conditions, care must be taken if a slotted or perforated stent is used to ensure that the particle size of the dispersed pharmaceutical powder, both the primary powder size and its aggregates and agglomerates, is small enough not to cause an irregular coating surface or to clog the slots or perforations of the stent. In cases where a dispersion is applied to the stent and it is desired to improve the smoothness of the coating surface or ensure that all particles of the composition of this invention are fully encapsulated in the polymer, or in cases where it is desirable to slow the release rate of the composition, deposited either from dispersion or solution, a clear (polymer only) top coat of the same polymer used to provide controlled release of the composition or another polymer can be applied that further restricts the diffusion of the composition out of the coating.

The composition coats the exterior and interior surfaces of the stent and, as it solidifies, encapsulates these surfaces in the polymer/composition of the present invention formulation. The dried stent thus includes a coating of the composition of the present invention on its surfaces. Preferably, the immersion methods are adapted such that the solution or suspension does not completely fill the interior of the stent or block the orifice. Methods are known in the art to prevent such an occurrence, including adapting the surface tension of the solvent used to prepare the composition, clearing the lumen after immersion, and placement of an inner member with a diameter smaller than the lumen in such a way that a passageway exists between all surfaces of the stent and the inner member. An alternative to dipping the distal end of the stent is to spray-coat the exterior and interior surfaces with a vaporized form of the composition comprising the composition of the present invention.

In one embodiment, the matrix is chosen such that it adheres tightly to the surface of the stent or synthetic graft. This can be accomplished, for example, by applying the matrix in successive thin layers. Each layer of matrix may incorporate a composition of this invention. Alternatively, composition of the present invention may be applied only to the layer in direct contact with the vessel lumen. Different types of matrices may be applied successively in succeeding layers.

The solvent is chosen such that there is the proper balance of viscosity, deposition level of the polymer, solubility of the pharmaceutical composition of this invention, wetting of the stent and evaporation rate of the solvent to properly coat the stents. In the preferred embodiment, the solvent is chosen such the composition and the polymer are both soluble in the solvent. In some cases, the solvent must be chosen such that the coating polymer is soluble in the solvent and such that the composition is dispersed in the polymer solution in the solvent. In that case the solvent chosen must be able to suspend small particles of the composition without causing them to aggregate or agglomerate into collections of particles that would clog the slots of the stent when applied. Although the goal is to dry the solvent completely from the coating during processing, it is a great advantage for the solvent to be non-toxic, non-carcinogenic and environmentally benign. Mixed solvent systems can also be used to control viscosity and evaporation rates. In all cases, the solvent must not react with or inactivate the composition or react with the coating polymer. Preferred solvents include, but are not limited to, acetone, N-methylpyrrolidone (NMP), dimethyl sulfoxide (DMSO), toluene, xylene, methylene chloride, chloroform, 1,1,2-trichloroethane (TCE), various freons, dioxane, ethyl acetate, tetrahydrofuran (THF), dimethylformamide (DMF), dimethylacetamide (DMAC), water, and buffered saline.

In one embodiment, a stent is coated with a mixture of a pre-polymer, cross-linking agents and the composition of the present invention, and then subjected to a curing step in which the pre-polymer and cross-linking agents cooperate to produce a cured polymer matrix containing the composition of the present invention. The curing process involves evaporation of the solvent and the curing and cross-linking of the polymer. Certain silicone materials can be cured at relatively low temperatures, (i.e., room temperature to 50° C.) in what is known as a room temperature vulcanization (RTV) process. Of course, the time and temperature may vary with particular silicones, cross-linkers and biologically active species.

Generally, the amount of coating to be placed on the catheter will vary with the polymer, and may range from about 0.1 to 40 percent of the total weight of the catheter after coating. The polymer coatings may be applied in one or more coating steps depending on the amount of polymer to be applied.

d. Addition of the Composition of the Present Invention to the Matrix

The composition of the present invention can be incorporated into the matrix, either covalently or noncovalently, wherein the coating layer provides for the controlled release of the composition from the coating layer. A composition of the present invention may be incorporated into each layer of matrix by mixing the composition of the present invention with the matrix coating solution. Alternatively, the composition may be covalently or noncovalently coated onto the last layer of matrix that is applied to the medical device. The desired release rate profile of the composition of the present invention from the device can be tailored by varying the coating thickness, the radial distribution (layer to layer) of the composition, the mixing method, the amount of the composition, the combination of different matrix polymer materials at different layers, and the crosslink density of the polymeric material, as discussed below.

In one embodiment, the composition of the present invention is added to a solution containing the matrix. For example, the composition can be incubated with a solution containing a polymer at an appropriate concentration of the composition. It will be appreciated that the concentration of the composition will vary and that one of ordinary skill in the art could determine the optimal concentration without undue experimentation. The composition of the present invention/polymer mixture is then applied to the device by any of the methods described herein.

The ratio of the composition of the present invention to polymer in the solution will depend on the efficacy of the polymer in securing the composition onto the stent and the rate at which the coating is to release the composition into the tissue of the blood vessel. More polymer may be needed if it has relatively poor efficacy in retaining the composition of the present invention on the stent and more polymer may be needed in order to provide an elution matrix that limits the elution of a very soluble composition. A wide ratio of composition of the present invention to polymer could therefore be appropriate and could range from about 10:1 to about 1:100.

e. Deposition of the Composition of the Present Invention onto a Coated Stent

In another embodiment, a medical device of this invention such as a stent comprises at least one layer of the composition of the present invention deposited on at least a portion of a coating layer of the stent. If desired, a porous layer can be deposited over the composition of the present invention layer, wherein the porous layer includes a polymer and provides for the controlled release of the composition therethrough and further avoids degradation of the composition. Methods of coating a stent according to this embodiment is disclosed in U.S. Pat. No. 6,299,604, which is specifically incorporated herein by reference.

In yet another embodiment, the policosanol and statin components of the composition of the present invention are covalently coupled to the matrix. In one embodiment, the composition of the present invention can be covalently coupled to the matrix through the use of hetero- or homobifunctional linker molecules. The use of linker molecules in connection with the present invention typically involves covalently coupling the linker molecules to the matrix after it is adhered to the stent. After covalent coupling to the matrix, the linker molecules provide the matrix with a number of functionally active groups that can be used to covalently couple with the policosanol and statin components. The linker molecules may be coupled to the matrix directly (i.e., through the carboxyl groups), or through well-known coupling chemistries, such as, esterification, amidation, and acylation. For example, the linker molecule could be a polyamine functional polymer such as polyethyleneimine (PEI), polyallylamine (PALLA) or polyethyleneglycol (PEG). A variety of PEG derivatives, e.g., mPEG-succinimidyl propionate or mPEG-N-hydroxysuccinimide, together with protocols for covalent coupling, are commercially available from Shearwater Corporation, Birmingham, Ala. (See also, Weiner, et al., J. Biochem. Biophys. Methods, 45:211-219 (2000), incorporated herein by reference). It will be appreciated that the selection of the particular coupling agent may depend on the type of delivery vehicle used in the composition of the present invention and that such selection may be made without undue experimentation.

f. Coating a Stent with the Composition of the Present Invention

In yet another embodiment, a thin layer of the composition of the present invention is covalently or noncovalently bonded to the exterior surfaces of the stent. In this embodiment, the stent surface is prepared to molecularly receive the composition according to methods known in the art. If desired, a porous layer can be deposited over the composition layer, wherein the porous layer includes a polymer and provides for the controlled release of the composition therethrough and further avoids degradation of the composition.

5. Compounded Medical Devices

In an alternative embodiment of a medical device according to the invention, the composition of the present invention is provided throughout the body of the medical device by mixing and compounding the composition directly into the medical device polymer melt before forming the medical device. For example, the composition of the present invention can be compounded into materials such as silicone, rubber or urethane. The compounded material is then processed by conventional method such as extrusion, transfer molding or casting to form a particular configuration. The medical device resulting from this process benefits by having the composition dispersed throughout the entire medical device body. Thus, the composition is present at the outer surface of the medical device when the medical device is in contact with bodily tissues, organs or fluids.

The present invention also provides a kit comprising the therapeutic composition of the present invention and a suitable excipient as described herein and a set of instructions, generally written instructions, although electronic storage media (e.g., magnetic diskette or optical disk) containing instructions are also acceptable, relating to the use and dosage of the therapeutic composition of the present invention for the intended treatment. The instructions included with the kit generally include information as to dosage, dosing schedule, and route of administration for the intended treatment. The containers of the therapeutic composition of the present invention may be unit doses, bulk packages (e.g., multi-dose packages) or sub-unit doses.

The invention is further illustrated by the following non-limiting examples. All scientific and technical terms have the meanings as understood by one with ordinary skill in the art. The specific examples which follow illustrate the methods in which the compositions of the present invention may be prepared and are not to be construed as limiting the scope of the invention. The methods may be adapted to variation in order to produce compositions embraced by this invention but not specifically disclosed. Further, variations of the methods to produce the same compositions in somewhat different fashion will be evident to one skilled in the art.

### EXAMPLE 1

Tablets comprising a composition of the present invention are prepared as set out in Tables III-IX.

TABLE III

| Ingredient | amt/cap | function |
|---|---|---|
| Mevacor ® (lovastatin) | 40 mg | active |
| Policosanol | 20 mg | active |
| Calcium phosphate | 261.7 mg | base |
| Cellulose | 49.4 mg | tablet coating agent |
| Stearic acid | 23.8 mg | lubricant |
| Magnesium stearate | 6.8 mg | lubricant |
| Silicon dioxide | 9.4 mg | diluent |

TABLE IV

| Ingredient | Amt/cap | function |
|---|---|---|
| Lescol ® (fluvastatin) | 40 mg | active |
| Policosanol | 20 mg | active |
| Calcium phosphate | 261.7 mg | base |
| Cellulose | 49.4 mg | tablet coating agent |
| Stearic acid | 23.8 mg | lubricant |
| Magnesium stearate | 6.8 mg | lubricant |
| Silicon dioxide | 9.4 mg | diluent |

TABLE V

| Ingredient | amt/cap | function |
|---|---|---|
| Crestor ® (rosuvastatin) | 5 mg | active |
| Policosanol | 20 mg | active |
| Calcium phosphate | 261.7 mg | base |
| Cellulose | 49.4 mg | tablet coating agent |
| Stearic acid | 23.8 mg | lubricant |
| Magnesium stearate | 6.8 mg | lubricant |
| Silicon dioxide | 9.4 mg | diluent |

TABLE VI

| Ingredient | amt/cap | function |
|---|---|---|
| Pravachol ® (pravastatin) | 20 mg | active |
| Policosanol | 20 mg | active |
| Calcium phosphate | 261.7 mg | base |
| Cellulose | 49.4 mg | tablet coating agent |
| Stearic acid | 23.8 mg | lubricant |
| Magnesium stearate | 6.8 mg | lubricant |
| Silicon dioxide | 9.4 mg | diluent |

TABLE VII

| Ingredient | amt/cap | function |
|---|---|---|
| Zocor ® (simvastatin) | 40 mg | active |
| Policosanol | 20 mg | active |
| Calcium phosphate | 261.7 mg | base |
| Cellulose | 49.4 mg | tablet coating agent |
| Stearic acid | 23.8 mg | lubricant |
| Magnesium stearate | 6.8 mg | lubricant |
| Silicon dioxide | 9.4 mg | diluent |

TABLE VIII

| Ingredient | amt/cap | function |
|---|---|---|
| Baycol ® (cervastatin) | 0.4 mg | active |
| Policosanol | 20 mg | active |
| Calcium phosphate | 261.7 mg | base |
| Cellulose | 49.4 mg | tablet coating agent |
| Stearic acid | 23.8 mg | lubricant |
| Magnesium stearate | 6.8 mg | lubricant |
| Silicon dioxide | 9.4 mg | diluent |

TABLE IX

| Ingredient | amt/cap | function |
|---|---|---|
| Lipitor ® (atorvastatin) | 40 mg | active |
| Policosanol | 20 mg | active |
| Calcium phosphate | 261.7 mg | base |
| Cellulose | 49.4 mg | tablet coating agent |
| Stearic acid | 23.8 mg | lubricant |
| Magnesium stearate | 6.8 mg | lubricant |
| Silicon dioxide | 9.4 mg | diluent |

The present invention provides an antihyperlipidemic composition of policosanol and statin, which is effective in lowering cholesterol levels, and a method of lowering cholesterol levels by employment of such an pharmaceutical or dietary supplement composition, or by the simultaneous administration of the ingredients thereof, all having the highly advantageous characteristics and effects as described herein. In a preferred embodiment, the composition is formulated for oral delivery.

The foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims that follow. The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1. A composition comprising policosanol and 3-hydroxy-3-methylglutaryl co-enzyme A (HMG-CoA) reductase inhibitor.

2. The composition of claim 1, wherein said policosanol comprises a mixture of straight chain primary aliphatic alcohols from 20 to 36 carbons in length.

3. The composition of claim 2, wherein said mixture of straight chain primary aliphatic alcohols includes:

| | |
|---|---|
| 1-eicosanol (C-20) | 0-5% |
| 1-docosanol (C-22) | 0-5% |
| 1-tetracosanol (C-24) | 0-30% |
| 1-hexacosanol (C-26) | 5-30% |
| 1-heptacosanol (C-27) | 0-5% |
| 1-octacosanol (C-28) | 5-80% |
| 1-nonacosanol (C-29) | 0-5% |
| 1-triacontanol (C-30) | 5-40% |
| 1-dotriacontanol (C-32) | 1-25% |
| 1-tetratriacontanol (C-34) | 0-7% |
| 1-hexatriacontanol (C-36) | 0-5%. |

4. The composition of claim 2, wherein said mixture of straight chain primary aliphatic alcohols includes:

| | |
|---|---|
| 1-eicosanol (C-20) | 0-5% |
| 1-docosanol (C-22) | 0-5% |
| 1-tetracosanol (C-24) | 12-27% |
| 1-hexacosanol (C-26) | 13-28% |
| 1-heptacosanol (C-27) | 0-5% |
| 1-octacosanol (C-28) | 15-25% |
| 1-triacontanol (C-30) | 25-40% |
| 1-dotriacontanol (C-32) | 5-15% |
| 1-tetratriacontanol (C-34) | 0-5%. |

5. The composition of claim 1, wherein said HMG-CoA reductase inhibitor is a statin.

6. The composition of claim 5, wherein said statin is selected from the group consisting of lovastatin, fluvastatin, rosuvastatin, pravastatin, simvastatin, cervastatin, and atorvastatin.

7. The composition of claim 1, further comprising a pharmaceutically acceptable carrier, excipient or dilutant.

8. The composition of claim 7, in the form of a capsule, tablet, liquid or powder.

9. A method for treating or preventing hypercholesterolemia related diseases in a mammal, which comprises administering a pharmaceutically effective amount of a composition comprising policosanol and 3-hydroxy-3-methylglutaryl co-enzyme A (HMG-CoA) reductase inhibitor to said human or mammal.

10. A method for reducing total cholesterol and LDL-cholesterol and increasing HDL-cholesterol levels in a mammal, which comprises administering a pharmaceutically effective amount of a composition comprising policosanol and 3-hydroxy-3-methylglutaryl co-enzyme A (HMG-CoA) reductase inhibitor to said human or mammal.

11. A method for lowering LDL-cholesterol, total cholesterol, increasing HDL-cholesterol and improving LDL-cholesterol/HDL-cholesterol ratio in a mammal, which comprises administering a composition comprising policosanol and 3-hydroxy-3-methylglutaryl co-enzyme A (HMG-CoA) reductase inhibitor in a pharmaceutically acceptable amount to said human or animal.

12. The composition of claim 1 wherein said policosanol comprises at least one higher primary aliphatic alcohol selected from stright chain primary aliphatic alcohols having 20 to 36 carbon atoms, and said HMG-CoA reductase inhibitor is a statin selected from the group consisting of lovastatin, fluvastatin, rosuvastatin, pravastatin, simvastatin, cervastatin, and atorvastatin wherein said composition is further characterized by a combination of policosanol and said statin in a quantitative ratio from 100:1 to 0.01:1 by weight.

13. The composition of claim 12 wherein said policosanol comprises 1-tetracosanol, 1-hexacosanol, 1-octacosanol, 1-triacontanol, 1-dotriacontanol and 1-tetratriacontanol, and said composition is further characterized by a combination of policosanol and statin in a quantitative ratio from 10:1 to 0.10:1 by weight.

14. The composition of claim 13, wherein said policosanol has the following quantitative composition:

| | |
|---|---|
| 1-docosanol (C-22) | 0-5 wt % |
| 1-tetracosanol (C-24) | 0-30 wt % |
| 1-hexacosanol (C-26) | 5-30 wt % |
| 1-heptacosanol (C-27) | 5-10 wt % |
| 1-octacosanol (C-28) | 10-20 wt % |
| 1-nonacosanol (C-29) | 0-5 wt % |
| 1-triacontanol (C-30) | 5-40 wt % |
| 1-dotriacontanol (C-32) | 1-25 wt % |
| 1-tetratriacontanol (C-34) | 0 7 wt %; |

and said composition is further characterized by a combination of policosanol and statin in a quantitative ratio from 3:1 to 0.33:1 by weight.

15. A method of treating hypercholesterolemic diseases, total cholesterol, LDL-cholesterol, coronary heart disease (heart attacks and strokes), inflammation, immunoregulatory diseases, cardiovascular diseases, and/or neurodegenerative disorders in a patient, comprising delivering to said patient a composition comprising policosanol and 3-hydroxy-3-methylglutaryl co-enzyme A (HMG-CoA) reductase inhibitor in an amount effective to provide prevent and/or reduce hypercholesterolemic diseases, total cholesterol, LDL-cholesterol, coronary heart disease (heart attacks and strokes), inflammation, immunoregulatory diseases, cardiovascular diseases, and/or neurodegenerative disorders in said individual, wherein said composition is delivered to said patient as a controlled release composition.

16. The method of claim 15, wherein said controlled release composition comprises a flowable thermoplastic polymer composition comprising a biocompatible polymer, a biocompatible solvent, policosanol and HMG-CoA reductase inhibitor and said controlled release composition is delivered to a bodily tissue or fluid in said patient, wherein the amounts of the polymer and the solvent are effective to form a biodegradable polymer matrix containing policosanol and HMG-CoA reductase inhibitor in situ when said composition contacts said bodily fluid tissue or fluid.

17. The method of claim 16, wherein said polymer is a poly(alkylene glycol) or a polysaccharide.

18. The method of claim 15, wherein the composition further comprises a controlled release additive.

19. The method of claim 16, wherein said biocompatible polymer is selected from the group consisting of polylactides, polyglycolides, polyanhydrides, polyorthoesters, polycaprolactones, polyamides, polyurethanes, polyesteramides, polydioxanones, polyacetals, polyketals, polycarbonates, polyorthocarbonates, polyphosphazenes, polyhydroxybutyrates, polyhydroxyvalerates, polyalkylene oxalates, polyacrylates, polyalkylene succinates, poly(malic acid), poly(amino acids) and copolymers, terpolymers, cellulose diacetate, ethylene vinyl alcohol, and copolymers and combinations thereof.

20. The method of claim 16, wherein the polymer matrix releases policosanol and HMG-CoA reductase inhibitor by diffusion, erosion, or a combination of diffusion or erosion as the polymer matrix biodegrades in said patient.

21. The method of claim 16, wherein said policosanol and HMG-CoA reductase inhibitor are added to said polymer composition prior to administration such that said polymer matrix further contains said said policosanol and HMG-CoA reductase inhibitor.

22. The method of claim 15, wherein said controlled release composition is in film form.

23. The method of claim 22, wherein said film comprises polylactic acid, polyglycolic acid and mixtures and copolymers thereof.

24. A kit comprising a first container comprising a controlled release formulation of policosanol and an HMG-CoA reductase inhibitor in an amount effective to treat or reduce and/or prevent hypercholesterolemic diseases, total cholesterol, LDL-cholesterol, coronary heart disease (heart attacks and strokes), inflammation, immunoregulatory diseases, cardiovascular diseases, and/or neurodegenerative disorders.

25. The kit of claim 24, further comprising a puncture needle or catheter.

26. An article of manufacture comprising:
a stent body comprising a surface; and
a coating comprising at least one layer disposed over at least a portion of the stent body, wherein the said layer comprises a polymer film having policosanol and a HMG-CoA reductase inhibitor dispersed therein.

27. A method of reducing LDL-cholesterol levels comprising:
administering to a mammal a pharmaceutical composition in an amount that inhibits the conversion of HMG-CoA to mevalonate and acetate to acetyl CoA while not inhibiting with the biosynthesis of CoQ10.

28. The method of claim 27, wherein the conversion of HMG-CoA to mevelonate is inhibited by a statin.

29. The method of claim 27, wherein the conversion of acetate to acetyl CoA is inhibited by policosanol.

30. The composition of claim 4, wherein said HMG-CoA reductase inhibitor is a statin selected from the group of lovastatin, fluvastatin, rosuvastatin, pravastatin, simvastatin, cervastatin, and atorvastatin.

31. The composition of claim 5, comprising said statin in a daily dose range from 0.1 mg/day to 160 mg/day.

32. The composition of claim 5, wherein said statin is selected from the group consisting of lovastatin, fluvastatin, simvastatin or atorvastatin and is presentin a daily dose range of 1-160 mg/day.

33. The composition of claim 5, wherein said statin is selected from the group consisting of lovastatin, fluvastatin, simvastatin or atorvastatin and is presentin a daily dose in the range of 5-60 mg/day.

34. The composition of claim 5, wherein said statin is selected from the group consisting of lovastatin, fluvastatin, simvastatin or atorvastatin and is present in a daily dose range of 10-40 mg/day.

35. The composition of claim 5, wherein said statin is rosuvastatin and is present in a daily dose e range of 1-20 mg/day.

36. The composition of claim 5, wherein said statin is rosuvastatin, administered in a daily dose range of 2-8 mg/day.

37. The composition of claim 5, wherein said statin is rosuvastatin and is present in a daily dose range of 4-6 mg/day.

38. The composition of claim 5, wherein said statin is prevastatin and is present in a daily dose range of 1-80 mg/day.

39. The composition of claim 5, wherein said statin is prevastatin and is present in a daily dose range of 5-30 mg/day.

40. The composition of claim 5, wherein said statin is prevastatin, administered in a daily dose in the range of 10-20 mg/day.

41. The composition of claim 5, wherein said statin is cervastatin, administered in a daily dose in the range of 0.1-1.6 mg/day.

42. The composition of claim 5, wherein said statin is cervastatin and is present in a daily dose range of 0.1-0.6 mg/day.

43. The composition of claim 5, wherein said statin is cervastatin and is present in a daily dose in the range of 0.1-0.4 mg/day.

44. A sustained release preparation comprising a pharmaceutically active mixture of policosanol and 3-hydroxy-3-methylglutaryl co-enzyme A (HMG-CoA) reductase inhibitor.

45. A transdermal preparation designed to administer pharmaceutically effective amounts of policosanol and 3-hydroxy-3-methylglutaryl co-enzyme A (HMG-CoA) reductase inhibitor into the blood stream.

46. The transdermal preparation of claim 45, wherein the policosanol and 3-hydroxy-3-methylglutaryl co-enzyme A (HMG-CoA) reductase inhibitor are present in a concentration sufficient such that the transdermal delivery system has an input rate when applied to the skin sufficient to produce a pharmaceutically effective steady state plasma concentration in the patient.

47. A transdermal delivery system for application to the skin of a patient, comprising:
(a) a drug impermeable backing layer;
(b) an adhesive layer;
(c) a drug permeable membrane, wherein the membrane is positioned relative to the backing layer so as to form at least one drug reservoir compartment between the membrane and the backing layer; and
(d) a composition comprising policosanol and 3-hydroxy-3-methylglutaryl co-enzyme A (HMG-CoA) reductase inhibitor contained within the drug reservoir compartment in a concentration sufficient such that the transdermal delivery system has an input rate when applied to the skin sufficient to produce a pharmaceutically effective steady state plasma concentration in the patient.

48. The method of claim 15, wherein said controlled release composition comprises a transdermal delivery system containing a mixture of policosanol and 3-hydroxy-3-methylglutaryl co-enzyme A (HMG-CoA) reductase inhibitor and said method comprises applying the transdermal delivery system to the skin of a patient and maintaining the transdermal delivery system in contact with the skin for a time sufficient to provide a pharmaceutically effective steady state plasma concentration in the patient.

49. A subcutaneous implant comprising policosanol and a statin.

50. The subcutaneous implant of claim 50 wherein said implant is effective to release levels of policosanol and statin over an extended period of time when subcutaneously implant in a human or animal in need thereof.

51. A method for administering policosanol and a statin to a human or animal which comprises subcutaneously implanting either a biodegradeable or nonbiodegradable polymer comprising a mixture of policosanol and said statin.

52. The method of claim 15, comprising administering said controlled release composition subcutaneously to the patient.
